(19) United States
(12) Patent Application Publication
(10) Pub. No.: US 2009/0326644 A1
(43) Pub. Date: Dec. 31, 2009

(54) PLANAR STRUCTURE AND METHOD FOR PRODUCING A PLANAR STRUCTURE

(76) Inventor: Frank Willems, Moers (DE)

Correspondence Address: COLLARD & ROE, P.C., 1077 NORTHERN BOULEVARD, ROSLYN, NY, 11576, US

(21) Appl. No.: 12/311,952

(22) PCT Filed: Oct. 1, 2007
(86) PCT No.: PCT/DE2007/001762

§ 371 Date: Jun. 4, 2009

(30) Foreign Application Priority Data
Oct. 26, 2006 (DE) 10 2006 051 013.5

(51) Int. Cl. A61F 2/06; B32B 3/10; B32B 37/00

(52) U.S. Cl. 623/1.39; 428/198; 156/73.2

(57) ABSTRACT

The invention relates to a planar structure made of fibers adhered to each other in certain locations, characterized in that the adhesions and/or fibers are broken by an ultrasound treatment. Such planar structures are utilized particularly in the medical field as vascular prostheses or tissue patches.

The invention relates to a planar structure made from fibers that are bonded together at some points.

Such type planar structures are mainly used as vascular prostheses or as tissue patches in medical engineering.

The invention further relates to a method of manufacturing a planar structure in which the fibers are applied onto a carrier where they bond together, at least partially. Such a method of manufacturing planar structures is known from DE 28 06 030 for example. A microporous fine fibrillar structure is achieved by spinning polycarbonate urethane to microfibers from a solution by means of a nozzle. Fibrillae made in this way are wound on forms in several hundred layers at defined angles and are molten or bonded together in layers at their points of intersection so that vascular prostheses or tissue patches having a mechanically and biologically stable microporous structure are manufactured.

The inner side of the vascular prosthesis or of the tissue patch, which is turned toward the blood, is intended to have, as far as practicable, a surface with a fine structure, whereas the outer side may have a coarser surface structure which ensures secure growth of connective tissue in vascular prosthesis after implantation thereof.

Finally, the invention relates to a vascular prosthesis that is preferably manufactured according to the method of the invention. It has been found out that vascular prostheses can be manufactured using methods such as the one described in the document DE 28 06 030 and that tissue patches may also be manufactured with similar methods. Usually, such type planar structures have a microporous fine fibrillar structure made from biocompatible materials. It has however been found out that the finished planar structures that are utilized as tissue patches or vascular prostheses in case of a tissue defect are not compatible with natural tissue to the extent desired.

Hitherto, small-lumen vascular prostheses are not available since the development of such type vascular prostheses constitutes a big challenge. All attempts failed because the vascular prostheses made were at risk of premature wear due to thrombus deposit and hyperplasia.

In the literature, it is discussed that the patency is of paramount importance for the physiological compliance of the vascular prosthesis (Salacinski et al.: “The mechanical behaviour of vascular grafts”, Journal of Biomaterials Applications, Vol. 15, January 2001, Page 241 and followings, as well as Cardiovascular Materials”, Garth W. Hastings, 1991, Chapter 1, Page 1 to 16, “Mechanical Properties of Arteries and Arterial Grafts”, V. How.) In practice however, no method is known that is suited for providing artificially manufactured planar structures with the physiological properties required in medical science.

It is therefore the object of the present invention to provide a planar structure and a method of manufacturing such a planar structure wherein the planar structure has such a differentiated natural structure that a widely physiological, preferably axial and tangential, elasticity (compliance) is achieved.

This object is achieved with a planar structure made from fibers, which are bonded together at some points, and in which bonds and/or fibers are broken through an ultrasonic treatment.

The idea underlying the invention is that an ultrasonic treatment is suited to influence the elasticity, or rather the E module, of a vascular prosthesis or of a tissue patch and the patency rates at the planar structure. Frequency, intensity and duration of the ultrasonic treatment must hereby be adapted to the material used for manufacturing the planar structure in order to achieve a change in the nonwoven structure on the one side and to avoid damage to the nonwoven structure on the other side.

Depending on the case of application and on the dimensions and thickness of the planar structure, special conditions are fixed for ultrasonic treatment in order for the treated planar structure to obtain a widely physiological structure.

Ultrasonic treatment causes the fibers to move so that the fibers are caused to extend and that cracks occur on the fibers, which however not only depend on the type of ultrasounds applied thereon but also on the structure of the planar structure and of the discrete fibers.

An advantageous implementation of a planar structure provides for the break lines to be oriented so as to be statistically distributed. Whilst in breaks resulting from the extension of the planar structure the break lines are usually arranged transverse to the direction of extension, a strain due to ultrasonic treatment leads to undirected extension that results in a characteristic arrangement of the break lines when viewing the planar structure treated under a microscope.

Both the vascular prostheses and the tissue patches are planar structures having two sides of which the one side is the inner side and one side the outer side with regard to using the planar structure. An effect of benefit is achieved if the inner side of the planar structure is smoother than its outer side. In particular in connection with a specially adapted patency rate, this causes a thin autochthonous neointima to develop on the inner side of the vascular prosthesis. Thus, the inner side of the planar structures is intended to have, as far as practicable, a finely structured surface, whereas the outer side may have a coarser surface structure. The coarser surface structure facilitates ingrowth of connective tissue and, as a result thereof, secure location inside the body.

A particular advantage is obtained if the planar structure comprises an axial and tangential elasticity that is adapted to a tissue. It has been found out that the vascular prostheses or tissue patches treated with ultrasound have a flexible material structure that could not be achieved hitherto with corresponding workpieces that had not experienced this treatment. Moreover, the vascular prostheses and tissue patches made have a longitudinal and transverse elasticity corresponding to the natural tissue.

It is advantageous if the planar structure comprises a fine fibrillar structure. This leads to a special surface structure that promotes the adsorption of thrombocytes in a physiologically advantageous amount.

In practice, planar structures with fibrillae having a diameter of 0.5 to 100μ have proved efficient. Advantageously, a spacing of 0.5 to 100μ is advantageously provided.

In vascular prostheses, the extraordinary high compliance causes the pulse waves of the blood to propagate physiologically in the sense of a function similar to that of an air chamber, this appearing from the triphasic flow speed amplitude in canine carotid and femoral interponates. In such a vascular prosthesis, a laminar flow is thus advantageously maintained so that the caliber jump feared in known vascular prostheses is avoided. Moreover, blood-damaging turbulences at the anastomoses associated with neointima detachment, dead space and hyperplasia formation are avoided. The flexible material structure obtained with ultrasonic treatment provides the vascular prostheses and patches with a particularly good shape retention for optimum flow properties with good buckling stability when internal pressure is applied.

In terms of method, the object underlying the invention is achieved using a method for manufacturing a planar structure wherein fibers are applied onto a carrier where they bond together at least partially, the bonded fibers being treated with ultrasound.

In an advantageous implementation, it is provided that the fibers comprise a polycarbonate urethane. Thus, the fibers may form microporous, fine fibrillar structures made from biocompatible polycarbonate urethanes. Moreover, the fibers may comprise a copolymer, in particular of a polycarbonate urethane, or a polymer alloy, in particular with polycarbonate urethanes. Such type materials have proved particularly biocompatible in practice and are very well suited for ultrasonic treatment.

In particular for manufacturing vascular prostheses it is proposed that the carrier comprises a cylindrical surface. Accordingly, for the manufacturing of tissue patches, it is proposed that the carrier comprises a planar surface.

In a particularly advantageous implementation variant it is proposed that the carrier comprises an ultrasound generator. If the ultrasounds are delivered directly through the carrier, the planar structure is particularly intensively irradiated with ultrasound.

It is further proposed that the fibers are applied to the carrier whilst their surface is still sticky. It is particularly advisable to apply the fibers with a spray nozzle. Both for manufacturing patches and in particular for manufacturing vascular prostheses it is proposed to move the spray nozzle and the carrier towards each other during the application of the fibers. The spray nozzle may for example be led around the carrier or be caused to move up and down relative to the carrier. An advantageous method variant however provides for rotation of the carrier relative to the spray nozzle and advantageously also that the carrier, which is configured to be a cylindrical carrier, is moved in the direction of its longitudinal axis.

Tests showed that it is advantageous to extend the fibers bonded together. Precisely the combination of extending the fibers of the planar structure and of ultrasonic treatment opens multiple possibilities of change in order to act upon the compliance of the planar structure and to achieve imposed parameters.

To carry out the method it is proposed that fibers bonded together to form a cylinder are extended with an extension mandrel. Here, after having been manufactured, a cylindrical planar structure can be pulled on an extension mandrel or an extension mandrel is pulled through the cylindrical planar structure. For the manufacturing of patches it is proposed that fibers bonded together to form a planar surface are retained at their edges and are extended over an extension block.

It has been found out that it is advantageous if the bonded fibers are extended by 5% to 40%, preferably by 10% to 30%. Depending on the type of material of the polycarbonate urethane used and on the frequency and intensity of the ultrasound as well as on the extension parameters, the vascular prosthesis or the tissue patch returns almost completely to its initial length or it stays slightly extended by 3% to 5%. In accordance with a development of the invention, this remaining extension is taken into consideration in such a manner that, prior to treatment, the size of the pores of the vascular prosthesis or of the tissue patch is configured to be smaller by the extent of extension one expects the prosthesis or patch to keep. In particular in the case of vascular prostheses which have to have a certain pore size to promote cell ingrowth the pore size of the surface is consciously configured smaller during manufacturing so that it has the desired width after extension treatment.

Advantageous results have been obtained with a method by which the bonded fibers are first extended and then treated with ultrasound. It has shown that the elasticity or rather the E module of a vascular prosthesis or of a tissue patch made from biocompatible polycarbonate urethanes is substantially improved by extension to a degree of about 10% to 30% and by subsequent ultrasound treatment in the extended condition.

For many fields of application it has proved advantageous to wash the bonded fibers after ultrasonic treatment.

An advantageous way of conducting the method is also obtained by treating the bonded fibers in an ultrasound bath.

An advantageous embodiment forms a vascular prosthesis that is preferably manufactured according to the previous method and that has an inner vascular diameter of less than 40, preferably of less than 12 mm. Advantageous exemplary embodiments range from 4 to 6 mm, that is, they are greater than 3 mm.

An exemplary embodiment for treating a vascular prosthesis and an exemplary embodiment for treating tissue patches are shown in the drawing and will be discussed herein after. In said drawing:

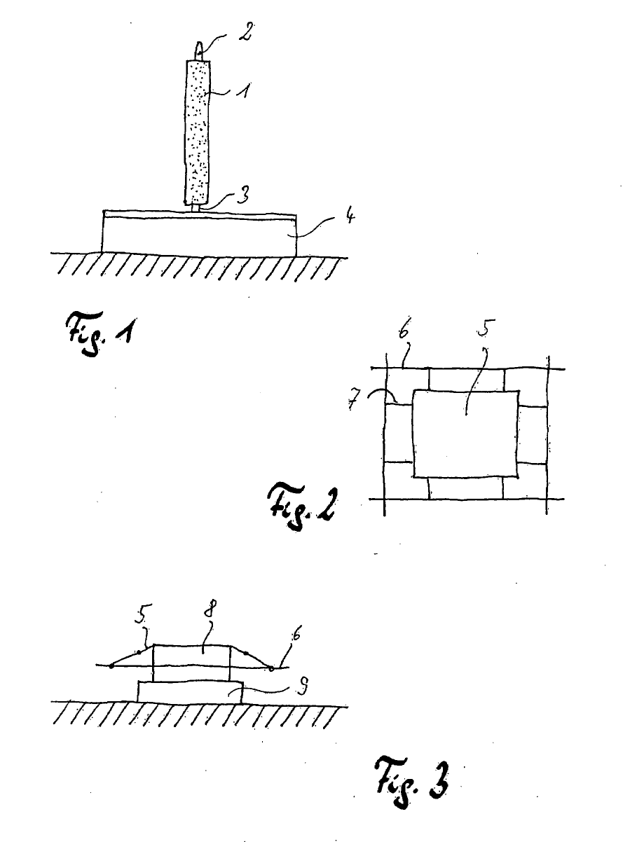

FIG. 1 schematically shows a vascular prosthesis placed on an extension mandrel,

FIG. 2 schematically shows a tissue path mounted in a frame,

FIG. 3 shows a tissue patch stretched over an extension block.

As shown in FIG. 1, the vascular prosthesis 1 is pulled for extension and for ultrasonic treatment over a mandrel 2 the outer diameter of which is about 10% to 30% greater than the inner diameter of the vascular prosthesis 1 to be treated. The surface 3 of the mandrel 2 has a very poor surface roughness in order to avoid the friction on the inner side of the vascular prosthesis 1 and the possible damages associated therewith.

After the vascular prosthesis 1 has been pulled onto the mandrel 2, ultrasound of a certain frequency and intensity is applied to said mandrel 2. For this purpose, the ultrasound generator 4 is used, which generates in the extension mandrel 2 ultrasonic vibrations that propagate to the vascular prosthesis 1. Through the ultrasound, the fibrillae (not shown) of the vascular prosthesis 1 are caused to vibrate. These vibrations can be so strong that fibrillae are destroyed. By purposefully setting the frequency and the intensity, all the fibrillae with a diameter smaller than a determined one can be destroyed. As a result, vascular prostheses 1 having defined elasticity or rather defined E module can be made.

The FIGS. 2 and 3 describe the treatment of tissue patches. For treating tissue patches with ultrasound, sheets of nonwoven fabric 5 manufactured during production are subjected to an extension degree of 10% to 30% by means of a rectangular frame 6. For this purpose, the sheet of nonwoven fabric 5 is pulled toward the frame 6 with the help of the threads 7. Next, the mounted sheet 5 is pressed onto a metal block 8, which has very poor surface roughness. The frame 6 is hereby pushed downward over the metal block 8 so that the threads 7 keep the sheet of nonwoven fabric 5 tense and extended.

Next, the metal block 8 is subjected to ultrasound of a certain frequency and intensity. Ultrasound causes the fibrillae to vibrate. By setting a certain frequency and intensity, the fibrillae having a diameter smaller than a certain diameter are destroyed. In the exemplary embodiment, the ultrasound generator 9 lies underneath the metal block 8 and transmits the ultrasound vibrations onto the sheet of nonwoven fabric 5 via the metal block 8.

The ultrasound generator however can also be the mandrel 2 or the metal block 8. Moreover, ultrasound insonification may also be performed in an ultrasound bath in which a liquid such as water conducts the ultrasound vibrations to the vascular prosthesis or to the sheet of nonwoven fabric.

The manufacturing of planar structures such as vascular prostheses or sheets of nonwoven fabric made from polymeric plastic materials is known to those skilled in the art and the published patent application DE 280 60 30 for example, which has been mentioned herein above, is fully incorporated herein by reference. In the exemplary embodiment, a planar structure made according to a method described in this published application is treated with ultrasound in order to manufacture a planar structure having a predetermined physiological compliance. A gel-like liquid made from dissolved granulates is sprayed hereby. As it is being sprayed, the solvent, the boiling point of which is less than 100° C., evaporates and fibers are obtained, which are deposited one above the other in layers in a spaced apart side-by-side relationship. A finished planar structure has between 50 and 300 layers.

1. A planar structure made from fibers that are bonded together at some points, wherein bonds and/or fibers are broken by ultrasonic treatment.

2. The planar structure as set forth in claim 1, wherein the break lines are oriented so as to be statistically distributed.

3. The planar structure as set forth in claim 1, wherein the planar structure comprises an inner side that is smoother than its outer side.

4. The planar structure as set forth in claim 1, wherein the planar structure comprises an axial and tangential elasticity that is adapted to a tissue.

5. The planar structure as set forth in claim 1, wherein the planar structure comprises a fine fibrillar structure.

6. The planar structure as set forth in claim 1, wherein the planar structure comprises fibrillae having a diameter ranging between 0.5 and 100μ.

7. The planar structure as set forth in claim 1, wherein the planar structure comprises fibrillae that are spaced from 0.5 to 100μ apart.

8. A method of manufacturing a planar structure wherein the fibers are applied to a carrier where they bond together at least partially, wherein the bonded fibers are treated with ultrasound.

9. The method as set forth in claim 8, wherein the fibers comprise a polycarbonate urethane.

10. The method as set forth in claim 8, wherein the fibers comprise a copolymer, in particular of a polycarbonate urethane.

11. The method as set forth in claim 8, wherein the fibers comprise a polymer alloy, in particular with a polycarbonate urethane.

12. The method as set forth in claim 8, wherein the carrier comprises a cylindrical surface.

13. The method as set forth in claim 8, wherein the carrier comprises a planar surface.

14. The method as set forth in claim 8, wherein the carrier comprises an ultrasound generator.

15. The method as set forth in claim 8, wherein the fibers are applied to the carrier whilst their surface is still sticky.

16. The method as set forth in claim 8, wherein the fibers are applied with a spray nozzle.

17. The method as set forth in claim 8, wherein spray nozzle and carrier are moved relative to each other during application.

18. The method as set forth in claim 8, wherein the bonded fibers are extended.

19. The method as set forth in claim 8, wherein fibers that are bonded to form a cylinder are extended with an extension mandrel.

20. The method as set forth in claim 8, wherein fibers bonded to form a planar surface are retained at their edges and are extended over an extension block.

21. The method as set forth in claim 8, wherein the bonded fibers are extended by 5% to 40%, preferably by 10%-30%.

22. The method as set forth in claim 8, wherein the bonded fibers are first extended and then treated with ultrasound.

23. The method as set forth in claim 8, wherein the bonded fibers are washed after ultrasonic treatment.

24. The method as set forth in claim 8, wherein the bonded fibers are treated in an ultrasound bath.

25. A vascular prosthesis, in particular manufactured according to the method, as set forth in claim 1, comprising an inner vessel diameter of less than 40, preferably of less than 12 mm.
